(19) United States
(12) Patent Application Publication
(10) Pub. No.: US 2023/0309962 A1
(43) Pub. Date: Oct. 5, 2023

(54) TELESCOPING CATHETER

(71) Applicant: BOSTON SCIENTIFIC SCIMED, INC., Maple Grove, MN (US)

(72) Inventors: Andy Nguyen, Minnetonka, MN (US); Monica Ortiz Serrano, Maple Grove, MN (US); Haruka Imura, Maple Grove, MN (US); Bryan Joseph Plunger, Minneapolis, MN (US)

(73) Assignee: BOSTON SCIENTIFIC SCIMED, INC., Maple Grove, MN (US)

(21) Appl. No.: 18/128,053

(22) Filed: Mar. 29, 2023

Related U.S. Application Data
(60) Provisional application No. 63/324,686, filed on Mar. 29, 2022.

(51) Int. Cl. A61B 8/00; A61B 8/08

(52) U.S. Cl. CPC A61B 8/445; A61B 8/0891; A61M 2025/0006

(57) ABSTRACT

Telescoping catheters for selectively moving a component of a medical device system are disclosed. An illustrative telescoping catheter may comprise a proximal hub adjacent a proximal end of the catheter, a drive cable, and a telescoping section extending from a proximal end adjacent the proximal hub to a distal end. The telescoping section may comprise an intermediate sheath having a proximal end coupled to the proximal hub and extending distally to a distal end, an outer sheath disposed over the intermediate sheath, the outer sheath having a proximal end and a distal end, an inner sheath disposed within the intermediate sheath, the inner sheath having a proximal end and a distal end, and a seal member disposed between the intermediate sheath and an outer surface of the inner sheath. The intermediate sheath may be configured to be longitudinally displaced relative to the inner and outer sheaths.

### CROSS-REFERENCE TO RELATED APPLICATIONS

This application claims the benefit of priority under 35 U.S.C. § 119 of U.S. Provisional Application No. 63/324,686, filed Mar. 29, 2022, the entire disclosure of which is hereby incorporated by reference.

### TECHNICAL FIELD

The present disclosure pertains to medical imaging, and systems and methods for medical imaging. More particularly, the present disclosure pertains to systems and methods for vascular imaging including a telescoping section.

### BACKGROUND

A wide variety of medical imaging systems and methods have been developed for medical use, for example, use in imaging vascular anatomy. Some of these systems and methods include intravascular imaging modalities. These systems and methods include various configurations and may operate or be used according to any one of a variety of methods. Of the known vascular imaging systems and methods, each has certain advantages and disadvantages. Accordingly, there is an ongoing need to provide alternative systems and methods for vascular imaging and assessment.

### BRIEF SUMMARY

This disclosure provides alternative telescoping catheters. In a first example, a telescoping catheter may comprise a proximal hub adjacent a proximal end of the telescoping catheter, a drive cable, and a telescoping section extending from a proximal end adjacent the proximal hub to a distal end. The telescoping section may comprise an intermediate sheath having a proximal end coupled to the proximal hub and extending distally to a distal end, an outer sheath disposed over the intermediate sheath, the outer sheath having a proximal end and a distal end, an inner sheath disposed within the intermediate sheath, the inner sheath having a proximal end and a distal end, and a seal member disposed between the intermediate sheath and an outer surface of the inner sheath. The intermediate sheath may be configured to be longitudinally displaced relative to the inner and outer sheaths.

Alternatively or additionally to any of the examples above, in another example, the seal member may be configured to form a fluid-tight seal between the inner sheath and the intermediate sheath.

Alternatively or additionally to any of the examples above, in another example, the intermediate sheath may have a constant outer diameter from the proximal end to the distal end.

Alternatively or additionally to any of the examples above, in another example, the seal member may comprise an o-ring.

Alternatively or additionally to any of the examples above, in another example, the outer sheath and the inner sheath may be fixed in relationship to each other.

Alternatively or additionally to any of the examples above, in another example, the intermediate sheath may define a lumen configured to substantially surround the inner sheath when the intermediate sheath is in a distally advanced configuration and surround a portion of the drive cable when the intermediate sheath is in a proximally displaced configuration.

Alternatively or additionally to any of the examples above, in another example, the telescoping catheter may further comprise an outer jacket disposed over the drive cable.

Alternatively or additionally to any of the examples above, in another example, the outer jacket may comprise a first layer and a second layer.

Alternatively or additionally to any of the examples above, in another example, the telescoping catheter may further comprise a housing coupled to the proximal end of the outer sheath.

Alternatively or additionally to any of the examples above, in another example, the telescoping catheter may further comprise a retainer sleeve secured to an outer surface of the intermediate sheath.

Alternatively or additionally to any of the examples above, in another example, the telescoping catheter may further comprise a retainer plug extending distally from the seal member.

Alternatively or additionally to any of the examples above, in another example, a proximal strain relief may be coupled to the proximal hub and may be configured to mechanically engage a proximal portion of the housing to limit proximal movement of the intermediate sheath.

Alternatively or additionally to any of the examples above, in another example, the retainer sleeve may be configured to mechanically engage a portion of the housing to limit proximal movement of the intermediate sheath.

Alternatively or additionally to any of the examples above, in another example, an outer diameter of the retainer plug may be the same as an outer diameter of the intermediate sheath.

Alternatively or additionally to any of the examples above, in another example, the retainer sleeve may be disposed over and contacting a distal end region of the intermediate sheath, the seal member, and a proximal end region of the retainer plug.

In another example, a telescoping catheter may comprise a drive cable and a telescoping section extending longitudinally along the telescoping catheter and having a distal end. The telescoping section may comprise an inner sheath coupled to the distal end of the telescoping section and defining a first lumen for housing the drive cable, an outer sheath coupled to the distal end of the telescoping section and defining a second lumen, wherein the outer sheath and the inner sheath may be fixed in relationship to each other, and the outer sheath substantially surrounds the inner sheath, an intermediate sheath having a proximal end and extending distally to a distal end, and a seal member positioned adjacent the distal end of the intermediate sheath and configured to provide a fluid tight seal between the intermediate sheath and the inner sheath. The intermediate sheath may be configured to slide between the inner sheath and the outer sheath for telescoping the telescoping catheter.

Alternatively or additionally to any of the examples above, in another example, the intermediate sheath may define a third lumen configured to substantially surround the inner sheath when the intermediate sheath is in a distally advanced configuration and surround a portion of the drive cable when the intermediate sheath is in a proximally displaced configuration.

Alternatively or additionally to any of the examples above, in another example, the telescoping catheter may further comprise a retainer sleeve secured to an outer surface of the intermediate sheath.

Alternatively or additionally to any of the examples above, in another example, the telescoping catheter may further comprise a retainer plug extending distally from the seal member.

Alternatively or additionally to any of the examples above, in another example, the intermediate sheath may have a constant outer diameter from the proximal end to the distal end.

Alternatively or additionally to any of the examples above, in another example, an outer diameter of the retainer plug may be the same as an outer diameter of the intermediate sheath.

Alternatively or additionally to any of the examples above, in another example, the retainer sleeve may be disposed over and contacting a distal end region of the intermediate sheath, the seal member, and a proximal end region of the retainer plug.

Alternatively or additionally to any of the examples above, in another example, the seal member may comprise an o-ring.

Alternatively or additionally to any of the examples above, in another example, the telescoping catheter may further comprise an outer jacket disposed over the drive cable.

Alternatively or additionally to any of the examples above, in another example, the outer jacket may comprise a first layer and a second layer.

In another example, a telescoping catheter may comprise a proximal hub adjacent a proximal end of the telescoping catheter, a drive cable, and a telescoping section extending from a proximal end adjacent the proximal hub to a distal end. The telescoping section may comprise a distal hub positioned adjacent to the distal end of the telescoping section, an intermediate sheath assembly, an outer sheath disposed over the intermediate sheath, the outer sheath having a proximal end and a distal end fixedly coupled to the distal hub, and an inner sheath disposed within the intermediate sheath, the inner sheath having a proximal end and a distal end fixedly coupled to the distal hub. The intermediate sheath assembly may comprise an intermediate sheath having a proximal end coupled to the proximal hub and extending distally to a distal end, a retainer plug having a proximal end and a distal end, the retainer plug extending colinear with the intermediate sheath, a seal member positioned between the distal end of the intermediate sheath and the proximal end of the retainer plug, and a retainer sleeve disposed over and contacting a distal end region of the intermediate sheath, the seal member, and a proximal end region of the retainer plug. The intermediate sheath assembly may be configured to be longitudinally displaced relative to the inner and outer sheaths.

Alternatively or additionally to any of the examples above, in another example, the retainer sleeve may be configured to mechanically engage a portion of a housing coupled to the proximal end of the outer sheath to limit proximal movement of the intermediate sheath.

Alternatively or additionally to any of the examples above, in another example, the intermediate sheath assembly may define a third lumen configured to substantially surround the inner sheath when the telescoping catheter is in a fully retracted configuration and surround a portion of the drive cable when the telescoping catheter is in a fully extended configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device;

FIG. 2 is a side view of another illustrative medical device.

FIG. 3 is a perspective view of the medical device of FIG. 2 in a retracted configuration;

FIG. 4 is a perspective view of the medical device of FIG. 2 in an extended configuration;

FIG. 5 is a cross-sectional view of a portion of the medical device of FIG. 2, taken at line 5-5 of FIG. 3;

FIG. 6 is a cross-sectional view of a portion of the medical device of FIG. 2, taken at line 6-6 of FIG. 3; and

FIG. 7 is a cross-sectional view of a portion of the medical device of FIG. 2, taken at line 7-7 of FIG. 4.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, or characteristics. However, such recitations do not necessarily mean that all embodiments include the particular features, structures, or characteristics. Additionally, when particular features, structures, or characteristics are described in connection with one embodiment, it should be understood that such features, structures, or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

In the medical arts, catheters are frequently used to diagnose and treat various disorders in a patient, such as, but not limited to, clogged or blocked blood vessels. A catheter is introduced into a blood vessel of a patient by, for example, making an incision in the patient over the blood vessel and inserting the catheter into the blood vessel of the patient. A catheter operator such as a physician then maneuvers the catheter through the blood vessels of the patient until the catheter is properly situated to diagnose or treat the disorder. Since maneuvering a catheter within a patient can sometimes be a difficult task, requiring a high degree of care and time taken to change the position of the catheter, a type of catheter known as a telescoping catheter is frequently used to perform these diagnoses and treatments. Disclosed herein are telescoping catheters with reduced telescope movement forces and reduced bubble formation.

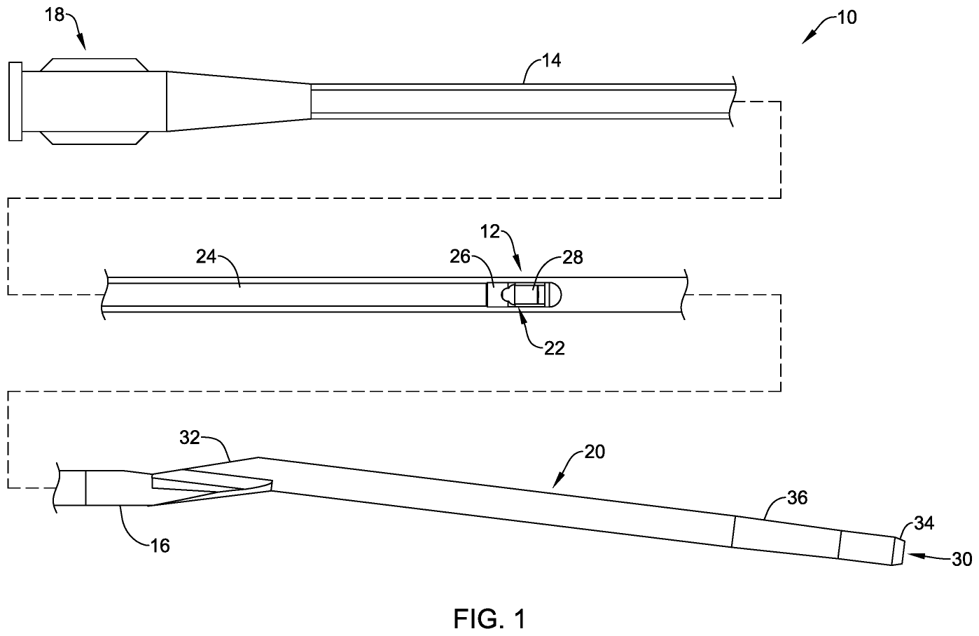

FIG. 1 is a side view of an example medical device 10. In at least some instances, the medical device 10 takes the form of an imaging medical device. For example, the medical device 10 may be an intravascular ultrasound (IVUS) device that may be used to image a blood vessel. The structure/form of the medical device 10 can vary. In some instances, the medical device 10 may include an elongate shaft 12 having a proximal end region 14 and a distal end region 16. A proximal hub or connector 18 may be coupled to or otherwise disposed adjacent to the proximal end region 14. A tip member 20 may be coupled to or otherwise disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate shaft 12. An imaging assembly 22 may be disposed within a lumen of the shaft 12. In general, the imaging assembly may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging assembly 22 may include a drive cable or shaft 24, a housing 26, and an imaging member or transducer 28 coupled to the drive cable 24 and/or housing 26. In at least some instances, the transducer 28 includes an ultrasound transducer. Other transducers are also contemplated. The transducer 28 may be rotatable and/or axially translatable relative to the shaft 12. For example, the drive cable 24 may be rotated and/or translated in order to rotate and/or translate the transducer 28 (and the housing 26).

While not explicitly shown in FIG. 1, the medical device 10 may include a telescoping section, configured to allow the medical device operator to move the drive shaft 24 including the imaging assembly 22 proximally and distally within the catheter, without having to move the entire catheter within the patient. This allows the catheter operator to easily change the location of the imaging assembly or other medical device within the patient. For example, the telescoping section may be actuated to change the location of the imaging assembly 22 within the elongate shaft 12. Disclosed herein are medical devices which reduce the telescope movement force required when actuating the telescoping portion.

Further, when using the medical device 10, it may be desirable to prepare and/or flush the shaft 12. In order to flush the medical device 10, fluid may be infused at a flush port on or at the hub 18. The fluid may exit the medical device at a vent hole (not shown) adjacent to the distal end of the housing 26. In some instances, the flushing process may result in the formation of bubbles within the shaft 12. It may be desirable to flush the medical device 10 in a manner that reduces the formation of bubbles and/or removes/disrupts any bubbles that are formed because bubbles may reflect/disrupt a signal (e.g., an ultrasound signal) from the transducer 28, which disrupts the image. While flushing is generally effective for removing bubbles, some bubbles may still get caught within the shaft 12. Disclosed herein are medical devices that are designed to help reduce the formation of bubbles within the medical device.

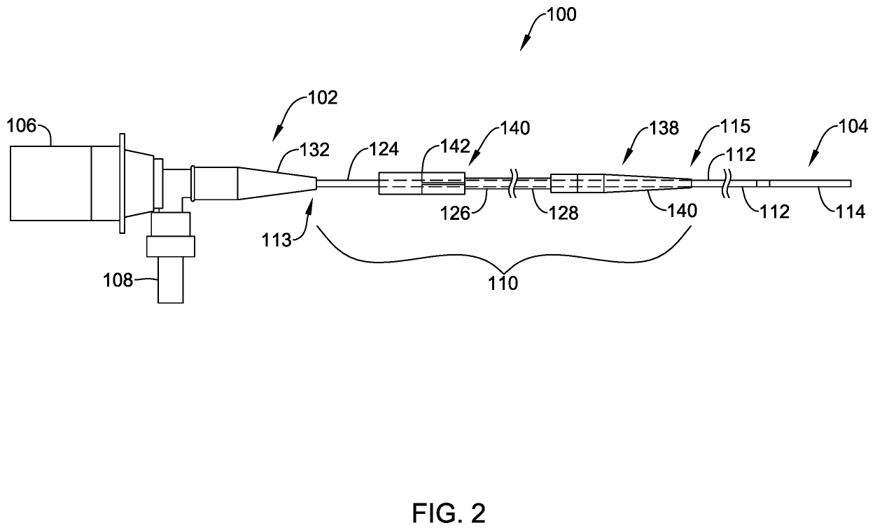

FIG. 2 illustrates a side view of another illustrative medical device, such as, but not limited to, a telescoping catheter 100. The catheter 100 extends from a proximal end region 102 to a distal end region 104. A proximal hub 106 may be affixed adjacent to the proximal end region 102. The proximal hub 106 may include a check valve and flush port 108. In order to flush the catheter 100, fluid may be infused at the flush port 108. The catheter 100 may further include a telescoping section 110 extending from a proximal end region 113 to a distal end region 115 and positioned between the proximal end region 102 and the distal end region 104 of the catheter 100. An elongate shaft 112 extends distally from the distal end region 115 of the telescoping section 110. The elongate shaft 112 may include a tip member 114 adjacent the distal end region 104 of the catheter 100. The tip member 114 may be similar in form and function to the tip member 20 described with respect to FIG. 1. For example, the tip member 114 may include a guidewire lumen having a guidewire exit port, an atraumatic distal end, one or more radiopaque markers, and/or other features.

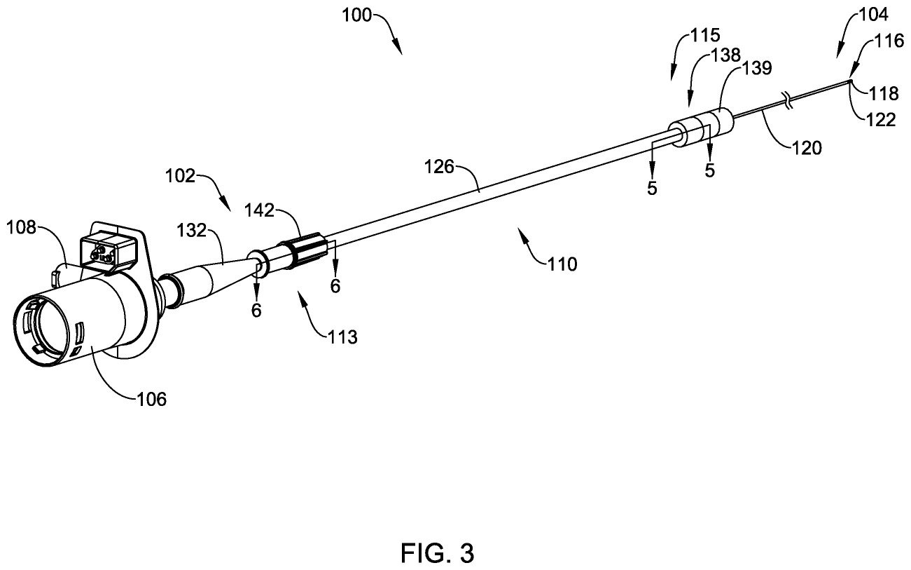

An imaging assembly 116 (see, for example, FIG. 3) may be movably positioned within a lumen of the elongate shaft 112. The imaging assembly 116 may include a drive cable or shaft 120, a housing 122, and an imaging member or transducer 118 coupled to the drive cable 120 and/or housing 122. It is contemplated that the imaging assembly 116 may include or be replaced with another medical device, such as, but not limited to, a cutting head, or other device. The particular device chosen for the drive cable 120 may be selected based on the desired function for the catheter 100. The drive cable 120 may extend proximally from the imaging member 118 through the telescoping section 110 to the proximal hub 106. The proximal hub 106 may contain components adapted to interface the drive cable 120 with a power source and/or other electronic couplings. In some cases, a proximal end of the drive cable 120 may be affixed to the proximal hub 106. While not explicitly shown, the drive cable 120 may include a single layer outer jacket or coating or a two-layer outer jacket or coating, as desired. If so provided, the outer jacket may extend a full length of the drive cable 120 or less than a full length of the drive cable 120.

The telescoping section 110 may include a first or intermediate sheath 124, a second or outer sheath 126, and a third or inner sheath 128. Generally, the outer sheath 126 may be disposed over the intermediate sheath 124 and the intermediate sheath 124 disposed over the inner sheath 128. The intermediate sheath 124 may be axially and/or rotatably displaced relative to the outer and inner sheaths 126, 128 such that movement of the proximal hub 106 is translated to movement of the intermediate sheath 124 and the drive cable 120. A distal hub 138 may be positioned adjacent the distal end region 115 of the telescoping section 110. The distal hub 138 may include a distal strain relief 139 configured to be coupled to the elongate shaft 112. Further, the distal ends of the outer sheath 126 and the inner sheath 128 may each be fixedly secured to the distal hub 138.

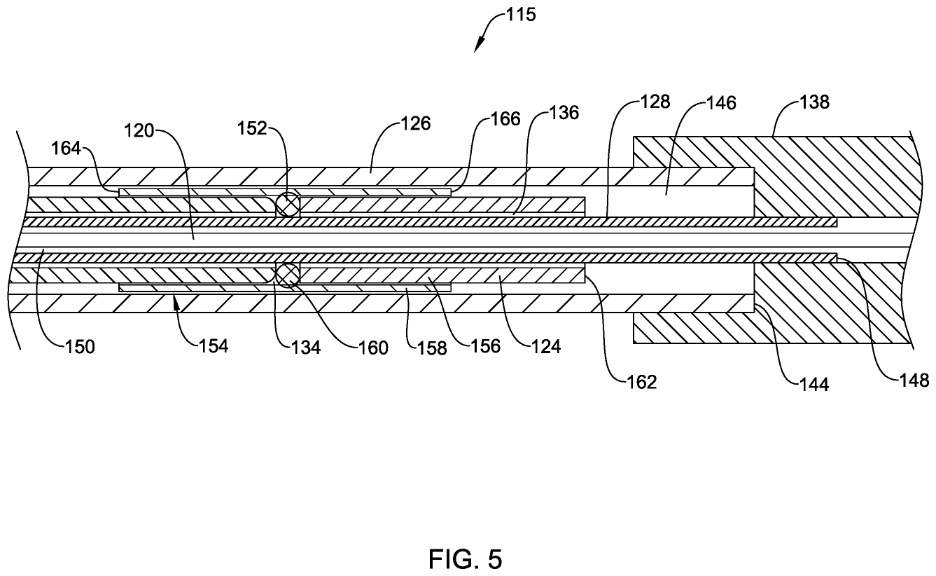

The intermediate sheath 124 extends distally from a proximal end region 130 coupled to a proximal strain relief 132 to a distal end 134 extending within the outer sheath 126. The intermediate sheath 124 may have a constant diameter from the proximal end region 130 to the distal end 134, although this is not required. The proximal strain relief 132 is coupled to the proximal hub 106. The intermediate sheath 124 is movable relative to the inner and outer sheaths 128, 126 such that the distal end 134 of the intermediate sheath 124 is movable between the distal hub 138 and a housing 142. The intermediate sheath 124 defines lumen 136 (see, for example, FIGS. 5-7) extending from the proximal end region 130 to the distal end 134 thereof. The lumen 136 may receive and/or house a portion of the drive shaft 120 and/or the inner sheath 128.

The outer sheath 126 extends distally from a proximal end region 140 coupled to a housing or receptacle 142 to a distal end 144 affixed to the distal hub 138 (see, for example, FIG. 5). The outer sheath 126 defines a lumen 146 (see, for example, FIGS. 5-7) extending from the proximal end region 140 to the distal end 144. The lumen 146 may receive or house a portion of the inner sheath 128 and/or the intermediate sheath 124.

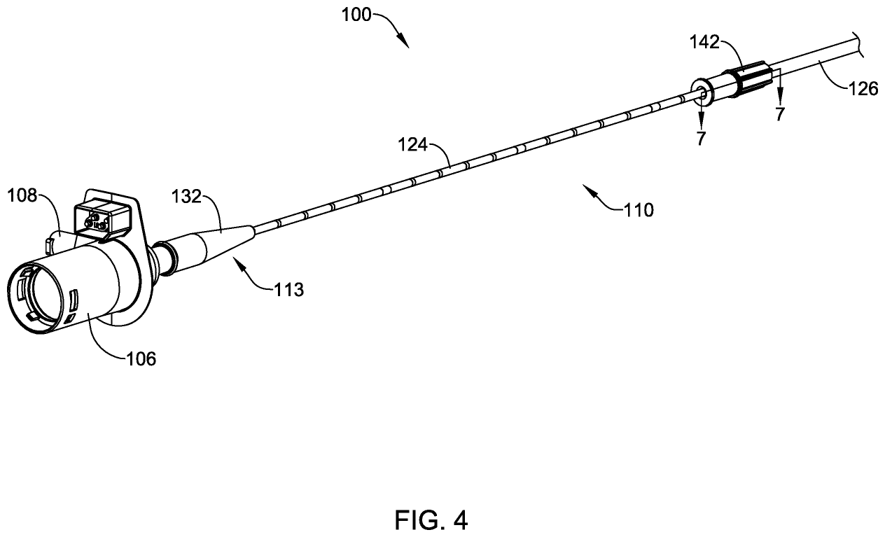

The inner sheath 128 extends distally from a proximal end region to a distal end 148 affixed to the distal hub 138 (see, for example, FIG. 5). The inner sheath 128 defines a lumen 150 (see, for example, FIGS. 5-7) extending from the proximal end region to the distal end 148. The lumen 150 may receive or house a portion of the drive shaft 120. For example, the inner sheath 128 may be configured to support the drive shaft 120 when the intermediate sheath 124 is in a proximally displaced configuration (see, for example, FIG. 4). In some embodiments, the proximal end region of the inner sheath 128 may be positioned adjacent to the proximal end region 140 of the outer sheath 126. In other embodiments, the proximal end region of the inner sheath 128 may be distal to the proximal end region 140 of the outer sheath 126.

FIG. 3 illustrates a perspective view of the telescoping catheter of FIG. 2 with the proximal hub 106 and the intermediate sheath 124 (and hence the drive shaft 120) in a distalmost position. This configuration may be considered to be fully retracted, as the catheter 100 has the shortest length. In FIG. 3, the elongate shaft 112 is not shown to more clearly show the structure of the imaging assembly 116. In the embodiment of FIG. 3, the intermediate sheath 124 has been distally advanced within the lumen 146 of the outer sheath 126. Distal movement of the intermediate sheath 124 may be limited by a mechanical stop created between the proximal strain relief 132 and the housing 142. When the proximal hub 106 and the intermediate sheath 124 are in a distalmost position, a majority of the length of the lumen 136 of the intermediate sheath 124 may surround the inner sheath 128.

FIG. 4 illustrates a perspective view of a proximal portion of the telescoping catheter of FIG. 2 with the proximal hub 106 and the intermediate sheath 124 (and hence the drive shaft 120) near a proximal most position. This configuration may be considered to be fully extended, as the catheter 100 has the greatest length. In the embodiment of FIG. 4, the intermediate sheath 124 has been proximally displaced within the lumen 146 of the outer sheath 126. Proximal movement of the intermediate sheath 124 may be limited by a mechanical stop created between mating features on a distal end region of the intermediate sheath 124 and the housing 142. When the proximal hub 106 and the intermediate sheath 124 are in a proximal most position, a majority of the length of the lumen 136 of the intermediate sheath 124 may surround the drive cable 120.

While FIGS. 3 and 4 illustrate the approximate extremes of the movement of the telescoping section 110, the proximal hub 106 and the intermediate sheath 124 may be positioned at any location between. As the drive cable 120 is coupled to the proximal hub 106, proximal and distal movement is translated to the drive cable 120 and the imaging assembly 116 to allow the imaging assembly to move without moving the entire catheter 100. It is further contemplated that rotational movement of the proximal hub 106 will also be translated to the drive shaft 120 and imaging assembly 116 to allow for rotation of the imaging assembly 116 within the elongate shaft 112.

FIG. 5 is a cross-sectional view of a distal end region 115 of the telescoping section 110 when the proximal hub 106 and the intermediate sheath 124 are in the distal-most configuration, taken at line 5-5 of FIG. 3. The drive shaft 120, inner sheath 128, intermediate sheath 124, and outer sheath 126 are co-axially arranged. In this configuration, the distal end 134 of the intermediate sheath 124 is adjacent or near to the distal ends 144, 148 of the outer and inner sheaths 126, 128. The telescoping section 110 further includes a seal member 152 positioned between the intermediate sheath 124 and an outer surface of the inner sheath 128 proximal to the distal end 134 of the intermediate sheath 124. The seal member 152 may be a flexible and/or deformable component configured to provide a fluid-tight seal between the intermediate sheath 124 and the inner sheath 128. In some cases, the seal member 152 may be a silicone o-ring. However, other seal member shapes and/or materials are contemplated. The seal member 152 may be sized and shaped to limit a fluid path when the telescoping catheter 100 is flushed. For example, when fluid is introduced via the port 108, fluid may fill the lumen 136 of the intermediate sheath 124 and the lumen 150 of the inner sheath 128. However, the seal member 152 prevents the fluid from exiting the lumen 136 of the intermediate sheath 124 and thus prevents the fluid from entering the lumen 146 of the outer sheath 126 providing a simpler flow path. Further, fluid is allowed to pass distally through the lumen 150 of the inner sheath 128, through the distal hub 138 and into the elongate shaft 112. It is further contemplated that positioning the seal member 152 between the intermediate sheath 124 and the inner sheath 128 may reduce the telescope movement force of the catheter 100.

In some embodiments, the intermediate sheath 124 may be a part of an intermediate sheath assembly 154. The intermediate sheath assembly 154 may include the intermediate sheath 124, a retainer plug 156, a retainer sleeve 158, and the seal member 152. The retainer plug 156 may extend co-linearly with the intermediate sheath 124. For example, the retainer plug 156 may have inner and outer diameters similar in size to the inner and outer diameters of the intermediate sheath 124, although this is not required. In some embodiments, the seal member 152 may be positioned between the distal end 134 of the intermediate sheath 124 and a proximal end 160 of retainer plug 156. However, this is not required. The seal member 152 may be positioned in other locations and/or configurations, as desired. For example, the seal member 152 may be configured to extend between an inner surface of the intermediate sheath 124 and/or the retainer plug 156 and an outer surface of the inner sheath 128. It is contemplated that the intermediate sheath 124 and the retainer plug 156 may be formed as separate components and subsequently attached. In other embodiments, the intermediate sheath 124 and the retainer plug 156 may be formed a single monolithic structure. For example, the intermediate sheath 124 and the retainer plug 156 may be molded as a single component over the seal member 152 or with a recess configured to receive the seal member 152. These are just some examples.

The retainer sleeve 158 may be positioned on an outer surface of the intermediate sheath 124, the retainer plug 156, and/or the seal member 152 and extend from a proximal end 164 to a distal end 166. The proximal end 164 of the retainer sleeve 158 may be positioned proximal to the distal end 134 of the intermediate sheath 124 while the distal end 166 of the retainer sleeve 158 may be positioned distal to the proximal end 160 of the retainer plug 156 such that the retainer sleeve 158 extends over a distal end region of the intermediate sheath 124, the seal member 152, and a proximal end region of the retainer plug 156. It is contemplated that the retainer sleeve 158 may be heat shrunk, molded, or adhered to the intermediate sheath 124, the seal member 152, and the retainer plug 156 In some embodiments, the retainer sleeve 158 may secure the intermediate sheath 124, the seal member 152, and the retainer plug 156 to one another. Additionally, or alternatively, the intermediate sheath 124, the seal member 152, and the retainer plug 156 may be secured using adhesives or formed as a unitary structure through molding or over-molding. The retainer sleeve 158 may have an inner diameter that is approximately the same as an outer diameter of the intermediate sheath 124 and/or the retainer plug 156 and an outer diameter that is less than an inner diameter of the outer sheath 126 such that the retainer sleeve 158 does not frictionally engage an inner surface of the outer sheath 126.

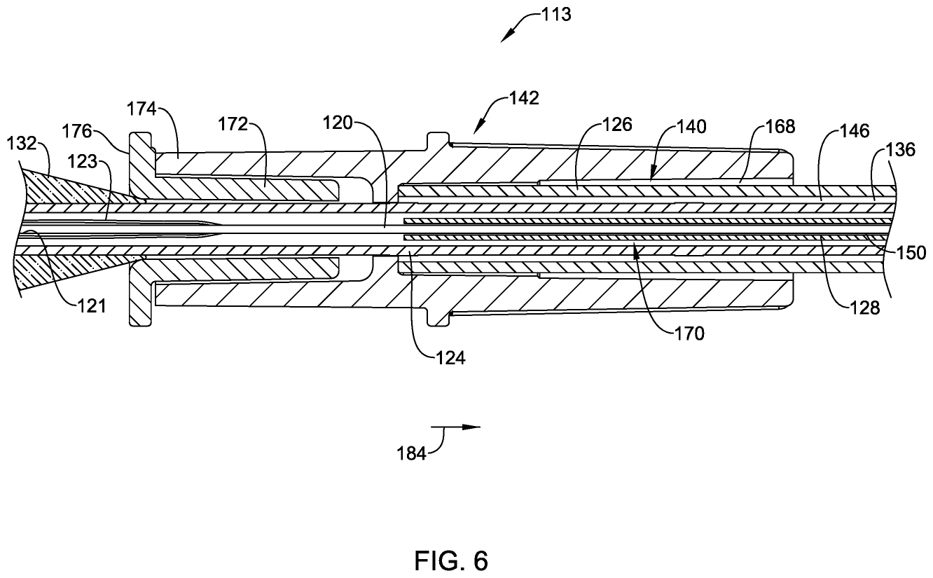

FIG. 6 is a cross-sectional view of a proximal end region 113 of telescoping section 110 when the proximal hub 106 and the intermediate sheath 124 are in the distal-most configuration, taken at line 6-6 of FIG. 3. The proximal end region 140 of the outer sheath 126 is secured within a lumen 168 of the housing 142. A proximal end region 170 of the inner sheath 128 may be free from attachment to other components. In some embodiments, the housing 142 may include an anchor seal plug 172 positioned at or near a proximal end 174 thereof. A proximal end 176 of the anchor seal plug 172 may be configured to provide a mechanical stop with the proximal strain relief 132 to limit further distal movement 184 of the proximal hub 106 and intermediate sheath 124. For example, when the proximal hub 106 and the intermediate sheath 124 are in the distal-most configuration, a distal end of the proximal strain relief 132 contacts the proximal end 176 of the anchor seal plug 172.

As noted above, the drive cable 120 may include an outer jacket or coating. The outer jacket may be formed from a first layer 121 and a second layer 123. However, in some embodiments, fewer than two (e.g., one or zero) layers may be provided or more than two layers (e.g., three or more) may be provided, as desired.

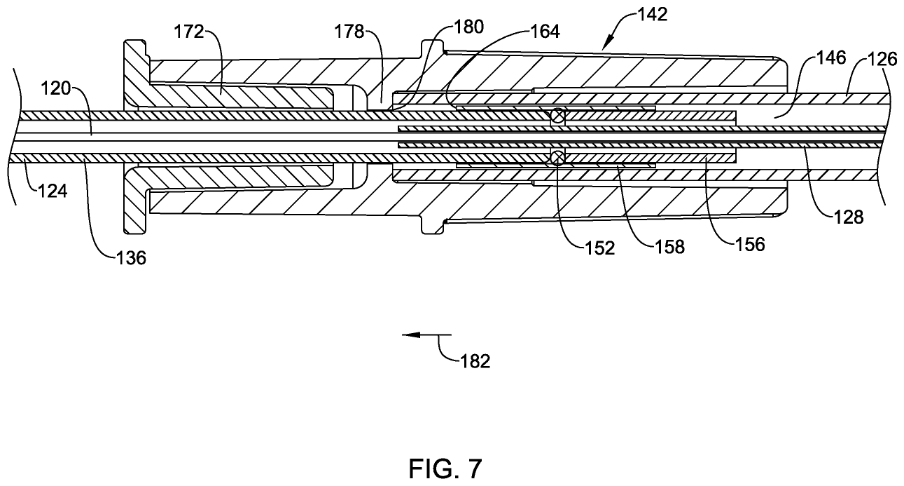

FIG. 7 is a cross-sectional view of an intermediate region of telescoping section 110 when the proximal hub 106 and the intermediate sheath 124 are near the proximal-most configuration, taken at line 7-7 of FIG. 4. In FIGS. 4 and 7, the proximal hub 106 and the intermediate sheath 124 have been actuated in the proximal direction 182 from the configuration illustrated in FIGS. 3, 5, and 6. In the illustrated embodiment, the proximal hub 106 and the intermediate sheath 124 have been proximally displaced until the proximal end 134 of the intermediate sheath 124 is disposed within the housing 142. The housing 142 may include an annular protrusion 178 extending radially inward from an inner surface of the housing 142. The annular protrusion may define a distal wall 180. It is contemplated that the protrusion 178 may define an opening having a diameter that is approximately the same as the outer diameter of the intermediate sheath 124. As the retainer sleeve 158 has an outer diameter greater than the intermediate sheath 124 further proximal displacement 182 of the proximal hub 106 and the intermediate sheath 124 causes the proximal end 164 of the retainer sleeve 158 to abut the distal wall 180 of the protrusion 178. This mechanical engagement limits proximal movement 182 of the proximal hub 106 and the intermediate sheath 124 and also prevents the intermediate sheath 124 from disengaging from the inner and outer sheaths 128, 126.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A telescoping catheter, the telescoping catheter comprising:
a proximal hub adjacent a proximal end of the telescoping catheter;
a drive cable;
a telescoping section extending from a proximal end adjacent the proximal hub to a distal end, the telescoping section comprising:
an intermediate sheath having a proximal end coupled to the proximal hub and extending distally to a distal end;
an outer sheath disposed over the intermediate sheath, the outer sheath having a proximal end and a distal end;
an inner sheath disposed within the intermediate sheath, the inner sheath having a proximal end and a distal end; and
a seal member disposed between the intermediate sheath and an outer surface of the inner sheath;
wherein the intermediate sheath is configured to be longitudinally displaced relative to the inner and outer sheaths.

2. The telescoping catheter of claim 1, wherein the seal member is configured to form a fluid-tight seal between the inner sheath and the intermediate sheath.

3. The telescoping catheter of claim 1, further comprising a retainer sleeve secured to an outer surface of the intermediate sheath.

4. The telescoping catheter of claim 1, wherein the intermediate sheath has a constant outer diameter from the proximal end to the distal end.

5. The telescoping catheter of claim 1, wherein the seal member comprises an o-ring.

6. The telescoping catheter of claim 3, further comprising a housing coupled to the proximal end of the outer sheath.

7. The telescoping catheter of claim 6, wherein the retainer sleeve is configured to mechanically engage a portion of the housing to limit proximal movement of the intermediate sheath.

8. A telescoping catheter, the telescoping catheter comprising:
a drive cable;
a telescoping section extending longitudinally along the telescoping catheter and having a distal end, the telescoping section comprising:
an inner sheath coupled to the distal end of the telescoping section and defining a first lumen for housing the drive cable;
an outer sheath coupled to the distal end of the telescoping section and defining a second lumen, wherein the outer sheath and the inner sheath are fixed in relationship to each other, and the outer sheath substantially surrounds the inner sheath;
an intermediate sheath having a proximal end and extending distally to a distal end; and
a seal member positioned adjacent the distal end of the intermediate sheath and configured to provide a fluid tight seal between the intermediate sheath and the inner sheath;
wherein the intermediate sheath is configured to slide between the inner sheath and the outer sheath for telescoping the telescoping catheter.

9. The telescoping catheter of claim 8, wherein the intermediate sheath defines a third lumen configured to substantially surround the inner sheath when the intermediate sheath is in a distally advanced configuration and surround a portion of the drive cable when the intermediate sheath is in a proximally displaced configuration.

10. The telescoping catheter of claim 8, further comprising a retainer sleeve secured to an outer surface of the intermediate sheath.

11. The telescoping catheter of claim 10, further comprising a retainer plug extending distally from the seal member.

12. The telescoping catheter of claim 11, wherein the intermediate sheath has a constant outer diameter from the proximal end to the distal end.

13. The telescoping catheter of claim 12, wherein an outer diameter of the retainer plug is the same as an outer diameter of the intermediate sheath.

14. The telescoping catheter of claim 11, wherein the retainer sleeve is disposed over and contacting a distal end region of the intermediate sheath, the seal member, and a proximal end region of the retainer plug.

15. The telescoping catheter of claim 10, wherein the seal member comprises an o-ring.

16. The telescoping catheter of claim 10, further comprising an outer jacket disposed over the drive cable.

17. The telescoping catheter of claim 16, wherein the outer jacket comprises a first layer and a second layer.

18. A telescoping catheter, the telescoping catheter comprising:
a proximal hub adjacent a proximal end of the telescoping catheter;
a drive cable;
a telescoping section extending from a proximal end adjacent the proximal hub to a distal end, the telescoping section comprising:
a distal hub positioned adjacent to the distal end of the telescoping section;
an intermediate sheath assembly comprising:
an intermediate sheath having a proximal end coupled to the proximal hub and extending distally to a distal end;
a retainer plug having a proximal end and a distal end, the retainer plug extending colinear with the intermediate sheath;
a seal member positioned between the distal end of the intermediate sheath and the proximal end of the retainer plug; and
a retainer sleeve disposed over and contacting a distal end region of the intermediate sheath, the seal member, and a proximal end region of the retainer plug;
an outer sheath disposed over the intermediate sheath, the outer sheath having a proximal end and a distal end fixedly coupled to the distal hub; and
an inner sheath disposed within the intermediate sheath, the inner sheath having a proximal end and a distal end fixedly coupled to the distal hub;
wherein the intermediate sheath assembly is configured to be longitudinally displaced relative to the inner and outer sheaths.

19. The telescoping catheter of claim 18, wherein the retainer sleeve is configured to mechanically engage a portion of a housing coupled to the proximal end of the outer sheath to limit proximal movement of the intermediate sheath.

20. The telescoping catheter of claim 18, wherein the intermediate sheath assembly defines a third lumen configured to substantially surround the inner sheath when the telescoping catheter is in a fully retracted configuration and surround a portion of the drive cable when the telescoping catheter is in a fully extended configuration.
